(19) United States
(12) Plant Patent Application Publication
(10) Pub. No.: US 2006/0075533 P1
(43) Pub. Date: Apr. 6, 2006

(54) Dianella caerulea plant named 'DCMP01'

(76) Inventor: Todd Anthony Layt, New South Wales (AU)

Correspondence Address: MYERS BIGEL SIBLEY & SAJOVEC, PO BOX 37428, RALEIGH, NC, 27627, US

(21) Appl. No.: 11/239,578

(22) Filed: Sep. 29, 2005

Related U.S. Application Data
(60) Provisional application No. 60/614,658, filed on Sep. 30, 2004.

(51) Int. Cl. A01H 5/00

(52) U.S. Cl. PLT/263

(57) ABSTRACT

‘DCMP01’ is a distinctive variety of Dianella caerulea which is characterized by the combination of its dense growth, slightly spreading and compact growth habit, purple-blue flower colour, and short canes with very short internodes, which is unusual for a Dianella caerulea, particularly when compared to the ‘Sydney Ecotype’. In addition, ‘DCMP01’ is less prone to falling over than the parent type.

### RELATED APPLICATION INFORMATION

This application claims the benefit of U.S. Provisional Application Ser. No. 60/614,658, filed Sep. 30, 2004, the disclosure of which is incorporated herein by reference in its entirety.

### LATIN NAME OF THE GENUS AND SPECIES

The Latin name of the novel variety disclosed herein is Dianella caerulea.

### VARIETY DENOMINATION

The inventive variety of Dianella caerulea disclosed herein has been given the varietal denomination ‘DCMP01’.

### BACKGROUND OF THE INVENTION

The present invention relates to a new and distinct perennial Blue Flax Lilly variety of Dianella caerulea, which has been given the varietal denomination of ‘DCMP01’. Its market class is that of an ornamental grass-like plant. ‘DCMP01’ is intended for use in landscaping and as a decorative grass-like plant.

An application for plant breeders' rights for variety ‘DCMP01’ has been filed with the Australian Plant Breeders' Rights Office, and was first gazetted in the Plant Varieties Journal in October 2003 under Application No. 2003/292.
Parentage:

The Dianella caerulea variety ‘DCMP01’ was first discovered in October 1996 in an Australian nursery in Clarendon, New South Wales, after an extensive breeding program. The ‘DCMP01’ parent, ‘Sydney Ecotype’ (unpatented), is characterized by a tall plant height, medium plant density, medium length aerial stems and yellow-green leaf colour.

In 1995, Dianella species were grown together in an open bed covering approximately 250 square metres of area. They were grown in groups of species and ecotypes including Dianella caerulea collected from the Sydney area, and other regions. The other Dianella species present were longifolia, revoluta and tasmanica. The plants were open pollinated with possible assisted pollination from general shaking of flower stems onto each other.

Seeds were collected and sown from the ‘Sydney Ecotype’ plants in December 1995. Approximately 30,000 plants were grown. In October 1996, using the selection criteria of compact growth habit and very short aerial stems (canes) with very short internodes, a single plant was identified as having these characteristics. This plant was selected, potted into a 140 mm pot for further evaluation and named ‘DCMP01’.
Asexual reproduction:

‘DCMP01’ was first asexually propagated by division in April 1997, in the state of New South Wales, Australia. It was asexually reproduced again during November 1997 and April 1998 and confirmed to be stable in character. The distinctive characteristics of the inventive variety, ‘DCMP01’, have remained stable and true to type from generation to generation through successive cycles of asexual reproduction including vegetative division and micropropagation.

### SUMMARY OF THE INVENTION

‘DCMP01’ is a distinctive variety of Dianella caerulea which is characterized by the combination of its dense growth, slightly spreading and compact growth habit, purple-blue flower colour, and short canes with very short internodes, which is unusual for a Dianella caerulea, particularly when compared to the ‘Sydney Ecotype’. In addition, ‘DCMP01’ is less prone to falling over than the parent type.

### BRIEF DESCRIPTION OF THE FIGURES

The photographs in the drawings were made using conventional techniques and show the colours as true as reasonably possible by conventional photography. Colours in the photographs may differ slightly from the colour values cited in the detailed botanical description, which accurately describe the colours of the new Dianella caerulea.

FIG. 1 shows a ‘DCMP01’ plant and illustrates the unusually short internodes located on short canes and a medium height flower spike that is characteristic of the variety.

FIG. 2 shows the variation in leaf width and glaucosity among the five Dianella caerullea varieties included in the comparison study.

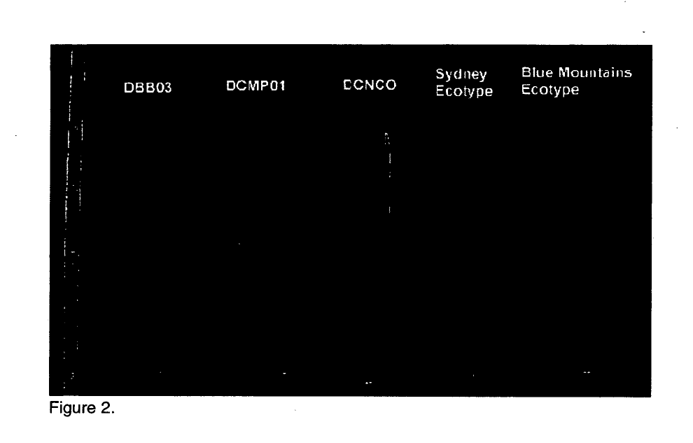

### BOTANICAL DESCRIPTION OF THE PLANT

The following is a detailed botanical description of a new and distinct variety of a Dianella caerulea ornamental grass-like plant known as ‘DCMP01’. The descriptions disclosed herein are based upon observations of the plant grown in 140 mm nursery pots and field plots in New South Wales, Australia. The plants were approximately 12-months-old at the time of observation. All colors cited herein refer to The Royal Horticultural Society Colour Chart (The Royal Horticultural Society, London, 2001 edition).

Those skilled in the art will appreciate that certain characteristics will vary with older or, conversely, younger plants. ‘DCMP01’ has not been observed under all possible environmental conditions. Where dimensions, sizes, colors and other characteristics are given, it is to be understood that such characteristics are approximations or averages set forth as accurately as practicable. The phenotype of the variety may differ with variations in the environment such as season, temperature, light intensity, day length, cultural conditions and the like.

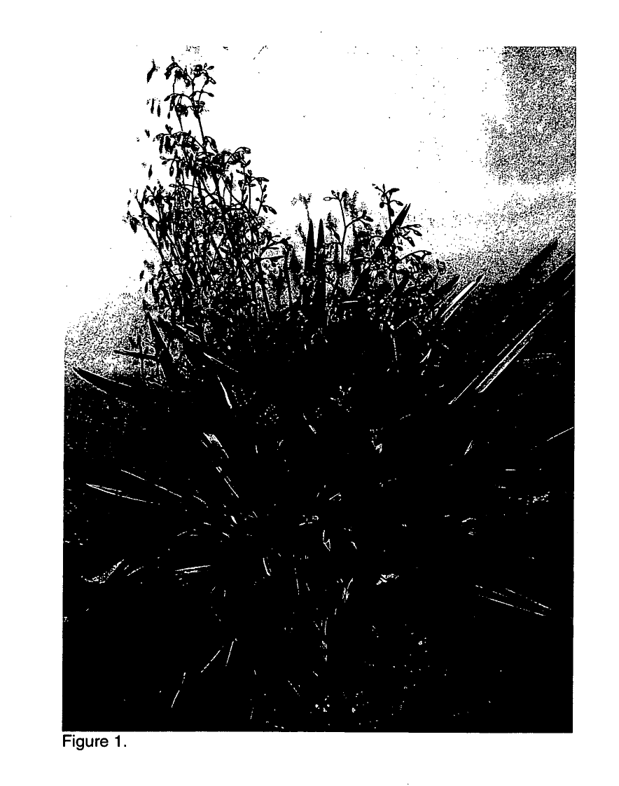

‘DCMP01’ is a perennial Dianella caerulea plant, which was produced through an innovative breeding program. After its selection, ‘DCMP01’ was asexually propagated by division and micropropagation. ‘DCMP01’ has dense growth, slightly spreading and compact growth habit, short rhizome form, purple-blue flower colour, unusually short internodes located on short canes, and a reduced tendency to fall over at maturity. A botanical description of ‘DCMP01’ and a comparison with other varieties of Dianella caerulea are provided below. A representative Dianella caerulea ‘DCMP01’ plant is shown in FIG. 1.
Technical description of the variety:
   Growth Habit.—‘DCMP01’ is a short, rhizomatous plant forming a semi-compact tussock. Its growth habit is erect, the height is short (mean 24.6 cm), and the density of shoots is strong.
   Stem.—Length of internodes is short.
   Foliage.—Leaf attitude erect, width medium (mean 12.2 mm), upper side colour with waxiness removed yellow-green (RHS 147A), lower side colour with waxiness removed yellow-green (RHS 147B), upper side glaucosity weak, shape ligulate, apex acute, cross section concave, spines on margin absent, spines on lower side midrib absent.
   Basal sheath.—Anthocyanin colour red-brown, intensity of anthocyanin colour weak.
   Inflorescence.—The inflorescence (flower spike) is purple-blue with a longer than average length when compared to Dianella caerulea ‘DCNCO’ (U.S. Provisional Application Ser. No. 60/614,804 filed Sep. 30, 2004; Australian Plant Breeders Rights Application No. 2003/293, received Oct. 8, 2003). The flower spike is soft to the touch.
   Environmental tolerances.—‘DCMP01’ has shown potential for shade tolerance. The winter hardiness of ‘DCMP01’ is at least to −10 degrees Celsius. ‘DCMP01’ has been observed to hold color to −6 degrees Celsius.

‘DCMP01’ has excellent drought tolerance compared with other Dianella caerulea varieties, most likely due to its tighter foliage. After severe wilting, ‘DCMP01’ has been noted to recover with watering.
Disease resistance and susceptibility.—‘DCMP01’ has excellent tolerance to pests and diseases and good resistance to root rot, which is a common problem in Dianella caerulea.

These features and other characteristics of the plant are apparent from the figures.
‘DCMP01’compared to other varieties of Dianella caerulea:

Grouping characteristics used to identify the most similar varieties of common knowledge to ‘DCMP01’ were short height and dense growth habit. Based on this, ‘DBB03’ (U.S. Provisional Application No. 60/614,654, filed Sep. 30, 2004; Australian Plant Breeders Rights Application No. 2003/291, received Oct. 8, 2003) was selected as the most similar suitable comparator for ‘DCMP01’. The parent (‘Sydney Ecotype’) and another form from the Blue Mountains region of New South Wales were also included in the trial. The comparative trial also included ‘DCNCO’, which has a much taller plant height than ‘DCMP01’.

The comparative trial of Dianella caerulea ‘DCMP01’ with other D. caerulea including ‘DBB03’, ‘DCNCO’, ‘Sydney Ecotype’, and Blue Mountains Ecotype’ (unpatented) was carried out in Summer 2002 through Autumn 2003 in Clarendon, New South Wales, Australia. The trial was conducted in open beds. The plants for this trial were propagated from divisions and planted into 130 mm pots filled with soilless potting mix. Nutrition was maintained with slow release fertilizers and pest and disease treatments were applied as required. The plants did not flower during the trial. Trial design included fifteen pots of each variety arranged in a completely randomized design. Measurements were taken from ten plants at random with one sample taken per plant.

In comparing ‘DCMP01’ with the other Dianella caerulea varieties, ‘DCMP01’ has a more compact habit than ‘Sydney Ecotype’, ‘Blue Mountains Ecotype’, ‘DBB03’ and ‘DCNC0’. ‘DCMP01’ generally has short internodes located on short canes, whereas canes are generally absent on ‘DBB03’ and ‘DCNC0’, and the ‘Sydney Ecotype’ has canes of typical length above the ground. ‘DCMP01’ has a taller flower spike height compared to ‘DCNC0’, but a shorter flower spike height compared to ‘DBB03’. The ‘Sydney Ecotype’ has a variable flower spike height. ‘DCNC0’ has a pale blue colour flower colour, whereas ‘DCMP01’ has a deeper blue almost purplish flower colour, and the ‘Sydney Ecotype’ has a variable flower colour. In addition, ‘DCMP01’ is easier and more productive to propagate from division than other known Dianella caerulea. Further comparisons are presented in Table I.

TABLE I
Comparison of Dianella caerulea varieties.

| | | | | |
|---|---|---|---|---|
| | | | | Blue |
| | | | Sydney | Mountains |
| ‘DBB03’ | ‘DCMP01’ | ‘DCNCO’ | ecotype | ecotype |

PLANT: GROWTH HABIT

| | | | | |
|---|---|---|---|---|
| erect | erect | erect | erect | semi- |
| | | | to semi- | erect to |
| | | | erect | erect |

PLANT HEIGHT (cm) LSD (P ≦ 0.01) = 6.03

| | | | | | |
|---|---|---|---|---|---|
| mean | 23.4 ^{c} | 24.6 ^{c} | 65.6 ^{a} | 63.0 ^{a} | 49.4 ^{b} |
| std deviation | 2.2 | 2.6 | 5.0 | 5.5 | 8.5 |

PLANT: DENSITY OF SHOOTS

| | | | | |
|---|---|---|---|---|
| strong | strong | medium- | medium | weak |
| | | strong | | |

STEM: INTERNODE LENGTH

| | | | | |
|---|---|---|---|---|
| short | short | short | long | very long |

LEAF: WIDTH (mm) LSD (P ≦ 0.01) = 1.97

| | | | | | |
|---|---|---|---|---|---|
| mean | 14.9 ^{c} | 12.2 ^{d} | 13.6 ^{c d} | 17.1 ^{b} | 22.2 ^{a} |
| std deviation | 2.1 | 0.6 | 0.8 | 0.9 | 2.9 |

LEAF: FOLIAGE COLOUR - overall appearance of leaf

| | | | | |
|---|---|---|---|---|
| blue- | yellow- | yellow- | yellow- | yellow- |
| green | green | green | green | green |

LEAF: COLOUR - waxiness removed

| | | | | | |
|---|---|---|---|---|---|
| upper side | 147A | 147A | 147B | 146B | ca 147A |
| lower side | 147A | 147B | 147B | 146B | 147B |

LEAF: GLAUCOSITY

| | | | | |
|---|---|---|---|---|
| strong | weak | weak | weak | weak |

LEAF: CROSS SECTION

| | | | | |
|---|---|---|---|---|
| concave | concave | slight | slight | concave |
| | | concave | concave | |

LEAF: PRESENCE OF SPINES ON MARGIN

| | | | | |
|---|---|---|---|---|
| absent | absent | present | present | present |

LEAF: PROMINENCE OF SPINES ON MARGIN

| | | | | |
|---|---|---|---|---|
| n/a | n/a | medium | medium | very weak |

LEAF: PRESENCE OF SPINES ON LOWER SIDE MIDRIB

| | | | | |
|---|---|---|---|---|
| absent | absent | present | present | present |

LEAF: PROMINENCE OF SPINES ON LOWER SIDE MIDRIB

| | | | | |
|---|---|---|---|---|
| n/a | n/a | medium | medium | medium |

BASAL SHEATH: COLOUR

| | | | | |
|---|---|---|---|---|
| blue- | red- | red- | brown | red to |
| green | brown | brown | to red- | red-brown |
| | | | brown | |

BASAL SHEATH: INTENSITY OF ANTHOCYANIN COLOUR

| | | | | |
|---|---|---|---|---|
| absent | weak | medium | medium | strong |

Mean values followed by the same letter are not significantly different at P ≦ 0.01 according to an S-N-K test.

That which is claimed is:

1. A new and distinct variety of Dianella caerulea plant named ‘DCMP01’, substantially as described and illustrated herein.
